(19) United States
(12) Patent Application Publication
(10) Pub. No.: US 2015/0353445 A1
(43) Pub. Date: Dec. 10, 2015

(54) METHOD FOR PERFORMING CARBON-CARBON COUPLING REACTIONS WITH CATIONIC EXCHANGE RESIN SUPPORTED PALLADIUM CATALYST

(71) Applicant: RHOM AND HAAS COMPANY, Philadlephia, PA (US)

(72) Inventors: Jose A. Trejo O' Reilly, Lansdale, PA (US); John Yamamoto, Doylestown, PA (US); James F. Tate, Jr, New Castle, DE (US)

(73) Assignee: Rhom and Haas Company, Philadelphia, PA (US)

(21) Appl. No.: 14/758,676

(22) PCT Filed: Jan. 27, 2014
(86) PCT No.: PCT/US14/13098

§ 371 Date: Jun. 30, 2015

Related U.S. Application Data
(60) Provisional application No. 61/759,435, filed on Feb. 1, 2013.

(51) Int. Cl. C07C 1/32

(52) U.S. Cl. CPC C07C 1/321; C07C 2531/08; C07C 2523/44

(57) ABSTRACT

A method for performing a carbon-carbon coupling reaction including the step of combining coupling reactants in the presence of a heterogeneous catalyst to yield a coupled reaction product, wherein the heterogeneous catalyst includes a palladium loaded strong acid cationic exchange resin.

### FIELD

The invention relates carbon-carbon coupling reactions catalyzed with strong acid cation exchange resins loaded with palladium.

### INTRODUCTION

Carbon-carbon coupling reactions are an important class of organic reactions. Coupling reactions involve a metal catalyzed “coupling” of two hydrocarbon fragments. Well known examples include: Suzuki, Heck, Sonogashira, Stille, Cassar, Buchwald-Hartwig, Kumada, Hiyama and Cadiot-Chodkiewicz coupling reactions. See for example: Colacot, The 2010 Nobel Prize in Chemistry: Palladium-Catalysed Cross-Coupling, Platinum Metals Review, 2011, (2), 84-90. A variety of polystyrene supported palladium catalysts have been described for use in coupling reactions. See for example: Basu, et al., Tetrahedron Lett. (2005), 46, 8591-8593; Lyubimov et al., Palladium-Containing Hypercrosslinked Polystyrene As An Easy To Prepare Catalyst For Suzuki Reaction In Water And Organic Solvents, Reactive & Functional Polymers 69 (2009) 755-758; Zhou et al., Reusable, Polystyrene-Resin-Supported, Palladium-Catalyzed, Atom Efficient Cross-Coupling Reaction of Aryl Halides with Triarybismuths, European Journal of Organic Chemistry (2010), 416-419; and US 2010/0119424. These catalysts include palladium supported by a non-functionalized crosslinked polystyrene adsorbent (e.g. DIAION™ HP20100) or anionic exchange resin (e.g. MACRONET™ MN100 and AMBERLITE™ IRA 900). Non-functionalized crosslinked polystyrene adsorbents generally require relatively high palladium loadings to be commercially viable, e.g. generally greater than 3 wt %. This high level of palladium is a major contributor to catalyst cost and palladium leaching. And while the lower palladium loadings have been reported with respect to anionic exchange resin, such resins are not typically thermally stable at coupling reaction temperatures of 80° C. or greater.

### SUMMARY

In a preferred embodiment, the invention provides an ion exchange support palladium catalyst with a higher palladium efficiency as compared with ion exchange supported palladium catalyst previously used in coupling reactions. In another preferred embodiment, the invention provides an ion exchange support palladium catalyst with a higher thermal stability than with ion exchange supported palladium catalyst previously used in coupling reactions. In yet another embodiment, the invention includes a method for performing a carbon-carbon coupling reaction including the step of combining coupling reactants in the presence of a heterogeneous catalyst to yield a coupled reaction product, wherein the invention is characterized by the heterogeneous catalyst including a palladium loaded strong acid cationic exchange resin. As many coupling reactions are conducted under basic conditions, the effectiveness of the subject catalyst (including a cationic polymer support) was unexpected.

### DETAILED DESCRIPTION

The invention includes a method for performing a carbon-carbon coupling reaction. Examples of coupling reactions are well known and include: Suzuki, Heck, Sonogashira, Stille, Cassar, Buchwald-Hartwig, Kumada, Hiyama and Cadiot-Chodkiewicz coupling reactions, and in particular Suzuki reactions (e.g. reaction between halide-substituted hydrocarbyl and a hydrocarbyl including a boronic acid functional group). The invention is particularly applicable to coupling reactions performed under basic reaction conditions (e.g. pH above 7, and preferably above 8). The invention is also suited for coupling reactions performed at temperatures of at least 80° C., 90° C. and even 100° C. (e.g. 90-200° C.). The method includes combining coupling reactants in the presence of a heterogeneous catalyst to yield a coupled reaction product. The method may be conducted in a single vessel as part of a batch or continuous process. The reactants may be provided in solution using a suitable solvent, e.g. methanol, toluene. The combination is heated and maintained at the appropriate coupling reaction temperature, e.g. 90° C. to 200° C., or 100° C. to 200° C.

The selection of coupling reactants is not particularly limited and includes those well known in the art. For example, Heck coupling reactants include halide-substituted arenes, e.g. including a benzene or pyridine moiety. Kumada coupling reactants include acetylenes with aryl of vinyl halides. Suzuki coupling agents including hydrocarbyl including a boronic acid functional group and a halide-substituted arenes, e.g. including a benzene or pyridine moiety. In a preferred embodiment, the coupling reactants include at least one of: a halide-substituted hydrocarbyl, an alkene or an alkyne. Representative reaction schemes are provided below:

Suzuki:

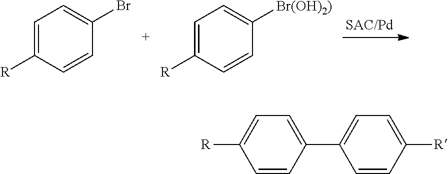

Heck:

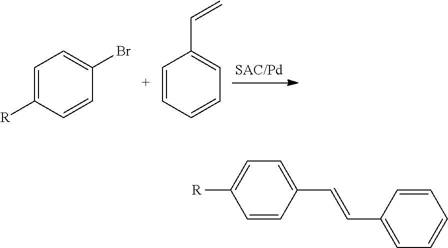

Stille:

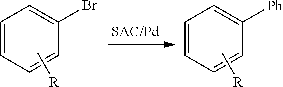

The heterogeneous catalyst of the present invention includes a strong acid cationic exchange resin loaded with palladium. Commercially available examples of such catalyst include AMBERLYST™ CH10, CH28 and CH43, all available from The Dow Chemical Company. These catalysts comprise a crosslinked polystyrene matrix including sulfonic acid functional groups. The crosslinked matrix is preferably a reaction product of a monomer mixture comprising styrene and divinylbenzene. The matrix preferably has a macro-reticular morphology and may be provided in a spherical bead form, which may have a uniform size distribution or Gaussian distribution, (as those terms are commonly understood in the ion exchange art). The beads preferably have an average particle diameter from 100 μm to 2 mm, more preferably from 150 μm to 1.5 mm and even more preferably from 250 to μm to 1 mm. The matrix preferably has a surface area from 10 to 100, preferably 15 to 75 and more preferably 20 to 50 m²/g. The matrix further preferably includes a total porosity of 0.1 to 0.9, preferably 0.2 to 0.7 and more preferably 0.25 to 0.5 cm³/g, with an average pore diameter of 50 to 2,500 Angstroms and preferably 150 to 1000 Angstroms.

The crosslinked copolymer matrix may be subsequently sulfonated by combining copolymer matrix with a sulfonating agent such as sulfuric acid, chlorosulfonic acid or sulfur trioxide, with or without a swelling agent. The reaction is preferably conducted at elevated temperature, e.g. 100-150° C. See for example: U.S. Pat No. 2,366,007, U.S. Pat. No. 2,500,149, U.S. Pat. No. 3,037,052, U.S. Pat. No. 5,248,435, U.S. Pat. No. 6,228,896, U.S. Pat. No. 6,750,259, U.S. Pat. No. 6,784,213, US 2002/002267 and US 2004/0006145—the entire contents of each of which is incorporated herein by reference. The resin preferably includes a sulfonic acid group content of 5.0 to 7.0, more preferably 5.1 to 6.5 and even more preferably 5.2 to 6. 0 meq/g, based on dry weight of polysulfonated cation exchange resin.

The resin is loaded with palladium. Applicable techniques for loading the resin with palladium ion are described in U.S. Pat. No. 6,977,314, US 2011/0124922, US 2012/0004468 and US 2012/0004468—the entire contents of which are each incorporated herein by reference. More specifically, the polysulfonated cation exchange resin may be loaded with palladium by contacting an aqueous solution of palladium ion with the hydrogen form of the cation exchange resin in a batch or column mode. Palladium ion may be provided in the salt form salt. The loaded cation exchange resin is then rinsed free of residual salts or acid. The amount of metal salt used is chosen such that the palladium or palladium ion will ultimately be present in an amount of about 1 to 15 g/L (0.1 to 2 wt % loading) of cation exchange resin, which can be determined by conventional analytical test methods. A representative loading method is as follows: 1 liter of polysulfonated cation ion exchange resin in hydrogen (H) form, is poured into a solution of 10-50 g of palladium nitrate in 0.5-2 liters of distilled water and the palladium is allowed to absorb onto the cation exchange resin for about 1 to 4 hours and then the solution is decanted from the resin. Alternatively, the polysulfonated cation exchange resin may be loaded with palladium by passing an aqueous solution of the metal salt through a column of the polysulfonated cation exchange resin until a desired level of palladium has been retained by the resin. This is followed by thorough washing with water to remove residual salts and acid generated during the loading process. Preferably, the catalyst is prepared by reducing a polysulfonated cation exchange resin containing palladium to deposit the palladium in elemental form in the catalyst. In this case the loaded resin may be subjected to ‘activation’ (reduction) by exposing the loaded resin to hydrogen (typically at room temperature and low partial pressures of hydrogen, for example, less than 1 bar). Alternatively, the activation may be conducted at temperatures up to about 120° C. and at hydrogen pressures of about 2 to 50 bar. The loaded catalyst containing the palladium in reduced form. The catalyst preferably comprises from 0.1 to 2 wt %, and more preferably 0.1 to 1 wt % of palladium, based on dry weight of cation exchange resin.

Many embodiments of the invention have been described and in some instances certain embodiments, selections, ranges, constituents, or other features have been characterized as being “preferred.” Characterizations of “preferred” features should in no way be interpreted as deeming such features as being required, essential or critical to the invention.

### EXAMPLES

### Example 1

### Strong Acid Cationic Exchange Resin (Macroreticular) Loaded with Palladium, Pd(II)

The following coupling reactants were charged within several batch reactors (R1-R4): catalyst, CsCO₃ (0.3 g), phenyl boronic acid (0.51 g), of bromobenzene (0.2 mL) and 4 mL solvent mixture of methanol and toluene (2 ml of each). The reactor was sealed and nitrogen purged three times and heated to 100° C. at 350 rpm and held at 0.6 MPa under nitrogen for 5 hours. The liquid was filtered and analyzed by GC-MS and results reported in Tables 1 a & 1b. The catalysts, both strong acid cationic resin (macroreticular) loaded with 0.7 wt % palladium, Pd(II) were AMBERLYST™ CH28 and CH43.

TABLE 1a
AMBERLYST ™ CH28
Catalyst Charged: AMBERLYST^{(TM)} CH28

| | | Reactor Identification Number | | | |
|---|---|---|---|---|---|
| | | R1 | R2 | R3 | R4 |
| Catalyst | g | 0.9103 | 0.5067 | 0.104 | 0.052 |
| Pd wt % dry basis | | 0.7 | 0.7 | 0.7 | 0.7 |
| GC-MS results: | | | | | |
| Benzene, bromo- | % | 3.30 | 1.70 | 3.60 | 5.40 |
| 1,1′-Biphenyl, 4-methyl- | % | 23.00 | 21.30 | 19.60 | 14.80 |
| 1,1′-Biphenyl, 4,4′-dimethyl- | % | 1.10 | 0.50 | 0.00 | 0.00 |
| Conversion | % | 85 | 92 | 84 | 75 |
| Selectivity | % | 95.4 | 97.7 | 100 | 100 |

TABLE 1a
AMBERLYST ™ CH43
Catalyst Charged: AMBERLYST^{(TM)} 46

| | | Reactor Identification Number | | | |
|---|---|---|---|---|---|
| | | R5 | R6 | R7 | R8 |
| Catalyst | g | 1.2351 | 0.5062 | 0.1143 | 0.558 |
| Pd wt % dry basis | | 0.7 | 0.7 | 0.7 | 0.7 |
| GC-MS results: | | | | | |
| Benzene, bromo- | % | 1.50 | 0.00 | 0.00 | 1.60 |
| 1,1′-Biphenyl,4-methyl- | % | 23.10 | 23.50 | 21.40 | 20.60 |
| 1,1′-Biphenyl,4,4′- | % | 1.30 | 0.60 | 0.00 | 0.00 |
| dimethyl- | | | | | |
| Conversion | % | 93 | 100 | 100 | 93 |
| Selectivity | % | 94.7 | 97.5 | 100 | 100 |

%-Selectivity=[Suzuki Coupling Product (1,1′ Biphenyl Benzene)]/[All Reaction Products]*100%

%-Conversion=100—(Initial %-w Bromobenzene)/(Final %-w Bromobenzene)*100

### Example 2

### Comparative Carbonaceous Resins Loaded with Palladium, Pd(0)

Batch reactors were charged with carbonaceous adsorbents, AMBERSORB™ 563 and AMBERLYST™ XN1010, both available from The Dow Chemical Company. Both resins were loaded with Pd to achieve 5% wt dry basis of Pd within the resin structure. The catalysts were reduced with H₂ at 110° C. for 24 hours under 0.6 MPa. After reduction, the reactors were open to siphon out the liquid and charged with the following coupling reactants: CsCO₃ (0.3 g), phenyl boronic acid (0.51 g), of bromobenzene (0.2 mL) and 4 mL solvent mixture of methanol and toluene (2 ml of each). The reactors were sealed and nitrogen purged three times and heated to 100° C. at 350 rpm and held at 0.6 MPa under nitrogen for 5 hours. The liquid was filtered and analyzed by GC-MS and results reported in Table 2.

TABLE 2
AMBERSORB ™ 563-Pd and AMBERLYST ™ XN1010-Pd:
Zero valent Pd examples.

| | | Catalyst Name | |
|---|---|---|---|
| | | AMBERSORB ™ | AMBERLYST ™ |
| | Units | 563-Pd | XN1010-Pd |
| Pd wt % dry basis | | 5 | 5 |
| Oxidation State of Pd | | 0 | 0 |
| Catalyst | G | 0.51 | 0.11 |
| Surface Area | m²/g | 538 | 570 |
| GC-MS results: | | | |
| Benzene, bromo- | % | 0.0% | 11.9% |
| 1,1′-Biphenyl | | 0.9% | 5.2% |
| 1,1′-Biphenyl, 4-methyl- | % | 8.3% | 4.9% |
| 1,1′-Biphenyl, 4,4′- | % | 0.7% | 0.8% |
| dimethyl- | | | |
| 1,1′:2′,1″-Terphenyl | % | Nd | 1.0% |
| Conversion | % | 100 | 45 |
| Selectivity | % | 83.4 | 43.52 |

%-Selectivity = [Suzuki Coupling Product (1,1′ Biphenyl Benzene)]/[All Reaction Products] * 100
%-Conversion = 100 − (Initial % − w Bromobenzene)/(Final % − w Bromobenzene) * 100

### Example 3

### Comparative Strong Base Anion Resin (Macroreticular) Loaded with Palladium

The Pd-impregnated resin (AMBERLITE™ 900, a strong base anion resin (macroreticular) loaded with 5 wt % palladium on a dry basis, available from The Dow Chemical Company) was reduced with sodium borohydrate and washed until neutral pH with 10 times the resin volume with deionized water. The following coupling reactants were charged in to the reactors: catalyst, CsCO₃ (0.3 g), phenyl boronic acid (0.51 g), of bromobenzene (0.2 mL) and 4 mL solvent mixture of methanol and toluene (2 ml of each). The reactor was sealed and nitrogen purged three times and heated to 100° C. at 350 rpm and held at 0.6 MPa under nitrogen for 5 hours. The liquid was filtered and analyzed by GC-MS and results reported in Table 3. The reaction conversion was obtained following the disappearance of bromobenzene and reported in % by comparing to the initial % wt.

TABLE 3

| | | Catalyst |
|---|---|---|
| | Units | AMBERLITE ™ 900-Pd |
| Resin type | | Anionic |
| Morphology | | Macroreticular |
| Pd loading | % wt dry basis | 0.7 |
| Resin Form - Counterion to | | Cl |
| quaternary sites: | | |
| Pd valence | | 0 |
| GC-MS results: | | |
| Bromo benzene | % | Nd |
| 1,1′ Biphenyl | % | 4.3 |
| 1,1′-biphenyl, 4-methyl | % | 3.4 |
| 1,1′-biphenyl, 4,4′-dimethyl- | % | 0.3 |
| Total biphenyls | % | 8.0 |
| Conversion | % | 100 |
| Selectivity | % | 54 |

Numbers reported are in %-Area to 100% of the total molecules identified.
%-Selectivity = [Suzuki Coupling Product (1,1′ Biphenyl Benzene)]/[All Reaction Products] * 100

### Example 4

### Strong Acid Cation Resin (Gellular) Loaded with Palladium

The following coupling reactants were charged in a batch reactor: catalyst, CsCO₃ (0.3 g), phenyl boronic acid (0.51 g), of bromobenzene (0.2 mL) and 4 mL solvent mixture of methanol and toluene (2 ml of each). The catalyst was AMBERLITE™ 121, a strong acid cation (gel) resin was loaded with 0.7 wt % Pd(II) on a dry basis. The reactor was sealed and nitrogen purged three times and heated to 100° C. at 350 rpm and held at 0.6 MPa under nitrogen for 5 hours. The liquid was filtered and analyzed by GC-MS and results reported in Table 4. The reaction conversion was obtained following the disappearance of bromobenzene and reported in % by comparing to the initial % wt.

TABLE 4

| | | Catalyst |
|---|---|---|
| | Units | AMBERLYST ™ 121-Pd |
| Resin type | | Cationic |
| Morphology | | Gellular |
| Pd loading | %-w dry basis | 0.7 |
| Resin Form - Counterion to | | H |
| quaternary sites: | | |
| Pd valence | | 2 |
| GC-MS results: | | |
| Bromo benzene | % | 8 |
| 1,1′ Biphenyl | % | 1.1 |
| 1,1′-biphenyl, 4-methyl | % | Nd |
| 1,1′-biphenyl, | % | 9.9 |
| 4,4′-dimethyl- | | |
| Total biphenyls | % | 100 |
| Conversion | % | 80 |
| Selectivity | % | 54 |

Numbers reported are in %-Area to 100% of the total molecules identified.
%-Selectivity = [Suzuki Coupling Product (1,1′ Biphenyl Benzene)]/[All Reaction Products] * 100.

### Example 5

### Various Commercial Catalysts vs. Strong Acid Cationic Resin (Macroreticular) Loaded with Palladium

The following coupling reactants were charged in to the reactors: catalyst, CsCO₃ (0.3 g), phenyl boronic acid (0.51 g), of bromobenzene (0.2 mL) and 4 mL solvent mixture of methanol and toluene (2 ml of each). The reactor was sealed and nitrogen purged three times and heated to 100° C. at 350 rpm and held at 0.6 MPa under nitrogen for 3.5 hours. The liquid was filtered and analyzed by GC-MS and results reported in the following. The catalysts tested were: EnCat30™ and EnCat40™ manufactured by Reaxa LTD and distributed through Sigma Aldrich Chemical; Activated Carbon/Pd (AC/Pd) available from Evonik Degussa; and Amberlyst™ CH28 available from The Dow Chemical Company.

TABLE 5

| | Blank | EnCat ™30 | EnCat ™40 | AC/Pd | Amberlyst ™ CH28 |
|---|---|---|---|---|---|
| Pd loading %-w | 0.00 | 4.30 | 4.30 | 5.00 | 0.70 |
| Bromo Benzene (%) | 21.66 | 4.42 | 5.40 | 6.32 | 6.26 |
| 1,1′ Biphenyl (%) | 0.00 | 23.74 | 26.42 | 30.76 | 22.86 |
| % Selectivity | | 99.00 | 99.00 | 99.00 | 99.00 |
| % Conversion | | 79.59 | 75.09 | 70.82 | 71.10 |
| Yield per Pd usage | | 5.52 | 6.14 | 6.15 | 32.66 |

1. A method for performing a carbon-carbon coupling reaction comprising the step of combining coupling reactants in the presence of a heterogeneous catalyst to yield a coupled reaction product,
wherein the invention is characterized by the heterogeneous catalyst comprising a palladium loaded strong acid cationic exchange resin.

2. The method of claim 1 wherein the heterogeneous catalyst comprises from 0.1 to 2 wt % palladium.

3. The method of claim 1 wherein the heterogeneous catalyst comprises from 0.1 to 1 wt % palladium.

4. The method of claim 1 wherein the strong acid cationic exchange resin comprises a macro reticular, crosslinked polystyrene matrix including sulfonic acid functional groups.

5. The method of claim 1 wherein the coupling reactants are maintained at a temperature of at least 80° C. in the presence of the heterogeneous catalyst.

6. The method of claim 1 wherein the coupling reactants comprise at least one of a halide-substituted hydrocarbyl, an alkene or an alkyne.

7. The method of claim 1 wherein the coupling reactants comprise a halide-substituted arene.

8. The method of claim 7 wherein the halide-substituted arene comprises at least one of a benzene or pyridine moiety.

9. The method of claim 1 wherein the coupling reactants comprise an alkyne-substituted arene.

10. The method of claim 1 wherein the coupling reactants comprise a cycloamine.
